(19) United States
(12) Patent Application Publication
(10) Pub. No.: US 2026/0249251 A1
(43) Pub. Date: Aug. 27, 2026

(54) PURIFICATION PROCESS OF ETHYL FORMATE

(71) Applicant: Taicang Hushi Reagent Co., Ltd., Taicang (CN)

(72) Inventors: Ming Lv, Taicang (CN); Xin Sun, Taicang (CN); Yongqi Wang, Taicang (CN); Jianjiang Zhi, Taicang (CN)

(21) Appl. No.: 19/644,219

(22) Filed: Apr. 10, 2026

(30) Foreign Application Priority Data
Jun. 25, 2025 (CN) 202510858469.7

(51) Int. Cl. B01D 69/14; B01D 67/00; B01D 71/02; B01D 71/34; B01D 71/38; C07C 67/48; C07C 69/06

(52) U.S. Cl. CPC B01D 69/141; B01D 67/0011; B01D 67/0083; B01D 67/0086; B01D 67/0088; B01D 67/00931; B01D 67/0095; B01D 71/028; B01D 71/34; B01D 71/381; C07C 67/48; C07C 69/06; B01D 2323/21811; B01D 2323/2189; B01D 2323/56

(57) ABSTRACT

Provided is a purification process for ethyl formate, belonging to the field of separation and purification. The purification process includes: preparing a molecular sieve doped with aluminum on a surface; modifying the surface of the molecular sieve to obtain a modified molecular sieve, and then combining the modified molecular sieve with porous polymer to obtain a composite, and supporting the composite on a porous matrix to prepare a separation membrane, thereby obtaining a purification material for separation and purification. The process is low in energy consumption and high in separation factor, and a separation material is reusable, and low in use cost.

### TECHNICAL FIELD

The present disclosure belongs to the field of separation and purification, and in particular relates to a purification process for ethyl formate.

### BACKGROUND

Ethyl formate, as an important industrial raw material, can be widely used in numerous fields. For example, the ethyl formate can serve as a pharmaceutical intermediate for drug synthesis, be used industrially in the production of coatings and paints, act as an insecticidal fumigant in agriculture, and function as a flavor and fragrance in the food industry. Industrially, the ethyl formate is commonly produced by the esterification of formic acid and ethanol. The process mainly involves direct esterification of formic acid and ethanol under the catalysis of sulfuric acid, followed by neutralization, water washing, and distillation to obtain a final product. During the production, the product ethyl formate and unreacted ethanol may form an azeotrope under an atmospheric pressure, the azeotrope is difficult to separate by using a conventional distillation method, resulting in high energy consumption for the preparation of ethyl formate. Most of the ethyl formate obtained in existing extractive distillation studies also has a purity of approximately 90%, which does not exceed azeotropic composition of ethyl formate and ethanol, and requires the addition of entrainers or extractants. In addition, the separation of these additional additives itself may cause extra consumption and costs. In addition, due to high miscibility of ethanol and water, the ethyl formate also contains difficult-to-remove water, and a resulting ternary system increases the difficulty of purifying ethyl formate.

### SUMMARY

To solve the problems of large separation and purification of ethyl formate, high energy consumption, and low product purity in the prior art, the present disclosure provides an efficient and low-energy-consumption purification process for ethyl formate, where a material for purification has high strength and can be reused repeatedly. The technical solution is as follows.

A purification material for ethyl formate is prepared by the following procedure: preparing a molecular sieve doped with aluminum on a surface, modifying the surface of the molecular sieve to obtain a modified molecular sieve, and then combining the modified molecular sieve with porous polymer to obtain a composite, and supporting the composite on a porous matrix to prepare a separation membrane, thereby obtaining a purification material.

Further, the preparation of the molecular sieve includes the following steps:
a. placing tetrapropylammonium hydroxide in a sodium hydroxide solution and mixing uniformly, then adding tetraethyl orthosilicate and mixing uniformly, and stirring for 6 h to 10 h; after carrying out crystallization for 48 h to 72 h, thoroughly washing a product, and drying the washed product to obtain a precursor; and
b. dispersing the precursor in water, adding the sodium hydroxide solution and mixing thoroughly, adding sodium metaaluminate and mixing uniformly, and then carrying out crystallization for 10 h to 24 h; thoroughly washing a product, drying, and then calcining the product at 500° C. to 600° C. for 5 h to 8 h to obtain the molecular sieve.

Further, in step a, a concentration of the sodium hydroxide solution ranges from 0.15 mol/L to 0.17 mol/L; and a mass ratio of the tetrapropylammonium hydroxide to the tetraethyl orthosilicate is 1.2 to 1.5:1.

Further, in step b, a mass ratio of the precursor to sodium hydroxide is 1:0.08 to 0.15; and a mass ratio of the precursor to the sodium metaaluminate is 1:0.08 to 0.12.

Further, the crystallization is carried out at 160° C. to 190° C.

Further, the preparation of the purification material includes the following steps: carrying out surface grafting modification of the molecular sieve with γ-methacryloxypropyltrimethoxysilane to obtain a modified molecular sieve;
placing polyvinylidene fluoride in dimethyl sulfoxide and mixing uniformly, and then adding polyvinyl alcohol; in a nitrogen atmosphere, heating a system until polyvinyl alcohol is completely dissolved, and ultrasonically mixing for 3 to 5 h; then adding the modified molecular sieve, ultrasonicating until the system is homogeneous, adding azodiisobutyronitrile, and mixing thoroughly for 0.5 h to 1 h to obtain homogeneous solution; and
casting the homogeneous solution onto a matrix to form a film, allowing infiltration by standing for 1 h to 2 h, then freezing to obtain a frozen membrane material; fully replacing dimethyl sulfoxide in the frozen membrane material with water, and drying a wet membrane material after completion of displacement with hot air at 70° C. to 85° C.; and after drying, thoroughly washing the membrane material with hot water, and drying the washed membrane material to obtain a purification material.

Further, a mass ratio of the molecular sieve to the γ-methacryloxypropyltrimethoxysilane is 1:0.15 to 0.25; a mass ratio of the modified molecular sieve to the polyvinylidene fluoride is 1:2 to 4; a mass ratio of the polyvinylidene fluoride to the dimethyl sulfoxide is 1:1.5 to 3; and a mass ratio of the polyvinylidene fluoride to the polyvinyl alcohol is 1:0.1 to 0.2.

Further, the freezing is carried out at a temperature below −15° C. for 8 h to 12 h.

A purification process for ethyl formate using the purification material includes the following steps: loading the purification material into a membrane module, and controlling a feed solution at 35° C. to 45° C.; and after carrying out negative pressure permeation for 1 h to 3 h, heating the feed solution to below 60° C., and collecting permeate during the process.

Further, a feed flow rate ranges from 10 mL/min to 50 mL/min, the membrane is taken out and treated at 50° C. to 80° C. under −0.08 to −0.04 MPa for 1 h to 3 h to complete regeneration.

By employing the technical solution, the present disclosure has the following advantages.
1. Compared with distillation and extraction, the purification process provided by the present disclosure features low energy consumption. The purification material has stable properties and is easy to separate without introducing other liquid impurities, with high separation accuracy and high product purity. In addition, the purified material has high strength, excellent stability, and long service life, can be readily regenerated, and is suitable for industrial application.
2. The purification process provided by the present disclosure adopts stage-wise temperature increase. At a lower temperature, it is conducive to permeation of smaller water molecules; and at a higher temperature, molecular motion accelerates, leading to increased permeation of ethanol. At this time, the water content in the feed solution is lower, which reduces the competition between water and ethanol for channels, thereby increasing the permeate flux and decreasing a permeation probability of ethyl formate. The problems of reduced separation factor and high energy consumption caused by continuous permeation at a high temperature for the purpose of increasing flux are avoided.
3. The purification material provided by the present disclosure exhibits strong affinity for ethanol, and due to performance difference between the ethyl formate and the ethanol, the ethanol in the feed mixture preferentially permeates through the purification material, thereby separating the ethanol from the ethyl formate. In addition, the purification material provided by the present disclosure is characterized by resistance to swelling in the ethyl formate, so that the purification material has considerable mechanical strength and structural stability, and appropriate affinity and hydrophobicity endow the material with a high permeate flux and a separation factor.
4. Pore channels inside the molecular sieve in the purification material provided by the present disclosure are not completely modified. Compared with ethyl formate, as silanol groups present therein exhibit stronger interaction with ethanol, ethanol can enter the pore channels more readily, thereby achieving separation from the ethyl formate. Furthermore, aluminum oxide is introduced onto the surface of the molecular sieve, which can modify polarity of the surface of the molecular sieve, thereby altering the interaction between the membrane and composition to be separated and further improving the separation factor. In addition, the aluminum oxide is easier to attract ethanol that has a smaller molecular volume and contains alcoholic hydroxyl groups, further enhancing the separation factor.
5. According to the present disclosure, pores of the polymer in the purification material and pores of the molecular sieve are both matched with the ethanol, so that the ethanol and the water molecules can permeate the purification material easily, while the ethyl formate, which has a larger molecular volume, is more likely to be blocked outside. This physically promotes the separation of the ethyl formate from the difficult-to-remove impurities, featuring high reliability and strong selectivity.
6. The molecular sieve in the purification material provided by the present disclosure can not only be used for separation and purification of the ethyl formate, but also serve as a filler to improve strength of the membrane. In addition, the polymer around the molecular sieve can enable sufficient dispersion of the molecular sieve to isolate the interaction between the molecular sieves and promote uniform distribution of the molecular sieves, thereby maintaining a high separation factor while maintaining a high permeate flux, and preventing the decrease in separation factor caused by agglomeration or local defects. This solves the problem that the molecular sieves tend to agglomerate, resulting in a low mass fraction in the material and limited room for improvement of the separation factor.
7. According to the present disclosure, an aluminum-free silicon-based precursor is firstly prepared, the precursor is then subjected to surface alkali treatment to ionize part of the silicon on the precursor surface, which then undergoes ion exchange with aluminum based on solubility differences, followed by calcination, so that the aluminum content is high merely on local surface of the molecular sieve, allowing aluminum to directly come into contact with a feed solution to be separated, while preserving more active silicon-based sites for modification.
8. According to the present disclosure, pore formation is carried out by using polyvinyl alcohol and dimethyl sulfoxide together to form a polyvinylidene fluoride skeleton that enables sufficient contact between the feed to be separated and the molecular sieve. On the basis of the pores created by the dimethyl sulfoxide, a phase change of the polyvinyl alcohol caused by its viscosity variation from low to high temperatures is utilized. During thermal crosslinking under hot air drying, the connectivity of channels is maintained are promoted, pore collapse is reduced, and the formed pore channels are endowed with high permeate flux.
9. A polyvinylidene fluoride skeleton in the purification material provided by the present disclosure exhibits excellent solvent resistance and high mechanical strength in ethyl formate, which can greatly maintain a pore size and stability of the molecular sieve, and can avoid uncontrollable cracks between the molecular sieve and the polymer skeleton caused by swelling, thereby obtaining high separation index and excellent purification performance.

### DETAILED DESCRIPTION OF THE EMBODIMENTS

The following clearly and completely describes the technical solutions in the embodiments of the present disclosure with reference to the accompanying drawings in the embodiments of the present disclosure the described embodiments are merely a part rather than all of the embodiments of the present disclosure. Based on the embodiments in the present disclosure, all other embodiments obtained by those of ordinary skill in the art without making creative labor fall within the scope of protection of the present disclosure.
Embodiment 1:(1) 3 g of tetrapropylammonium hydroxide was added into sodium hydroxide solution with a concentration of 0.16 mol/L and uniformly mixed, then 2 g of tetraethyl orthosilicate was added, uniformly mixed, and then stirred for 8 h to obtain a mixture. The mixture was crystallized at 180° C. for 48 h to obtain a product, and then the product was thoroughly washed and dried to obtain a precursor. 1.5 g of the precursor was dispersed in water, and 10 mL of sodium hydroxide solution with a concentration of 0.4 mol/L was added and uniformly mixed, and then 0.15 g of sodium metaaluminate was added, uniformly mixed, and crystallized at 180° C. for 18 h to obtain a product. The product was thoroughly washed, dried, and then calcined at 550° C. for 6 h to obtain a molecular sieve.
(2) 1.2 g of molecular sieve was placed in 200 mL of methanol for ultrasonic dispersion, 60 mL of aqueous ammonia diluted by 50 times was added, and then 0.25 g of γ-methacryloxypropyltrimethoxysilane was added for reaction at 50° C. for 6 h to obtain a product, and the product was washed with ethanol and water for three times and then dried to obtain a modified molecular sieve.
(3) 3 g of polyvinylidene fluoride 17-88 was placed in 7 g of dimethyl sulfoxide and uniformly mixed, and then 0.45 g of polyvinyl alcohol was added. In a nitrogen atmosphere, a system was heated to 80° C., and after the polyvinyl alcohol was completely dissolved, ultrasonic mixing was carried out for 4 h. Subsequently, 1 g of modified molecular sieve was added, and ultrasonicating was carried out until the system is homogeneous. Then, azodiisobutyronitrile was added and thoroughly mixed for 0.5 h to obtain homogeneous liquid.
(4) The homogeneous liquid was cast onto a polytetrafluoroethylene matrix with a pore size of 0.2 μm to form a film, infiltrated by standing for 1 h, and then frozen below −18° C. for 12 h to obtain a frozen membrane material. The frozen membrane material was immersed into water, the dimethyl sulfoxide in the frozen membrane material was displaced with the water, and the immersion and displacement operations were repeated for five times. A wet membrane material after completion of displacement was dried with hot air at 70° C. to 85° C.; and after drying, the membrane material was thoroughly washed with hot water, and dried to obtain a purification material.
Embodiment 2: A difference from Embodiment 1 is that:
(2) 1.2 g of molecular sieve was placed in 200 mL of methanol for ultrasonic dispersion, 60 mL of aqueous ammonia diluted by 50 times was added, and then 0.18 g of γ-methacryloxypropyltrimethoxysilane was added for reaction at 50° C. for 6 h to obtain a product, and the product was washed with ethanol and water for three times and then dried to obtain a modified molecular sieve.
Embodiment 3: A difference from Embodiment 1 is that:
(2) 1.2 g of molecular sieve was placed in 200 mL of methanol for ultrasonic dispersion, 60 mL of aqueous ammonia diluted by 50 times was added, and then 0.3 g of γ-methacryloxypropyltrimethoxysilane was added for reaction at 50° C. for 6 h to obtain a product, and the product was washed with ethanol and water for three times and then dried to obtain a modified molecular sieve.
Embodiment 4: A difference from Embodiment 1 is that:
(3) 2 g of polyvinylidene fluoride 17-88 was placed in 7 g of dimethyl sulfoxide and uniformly mixed, and then 0.45 g of polyvinyl alcohol was added. In a nitrogen atmosphere, a system was heated to 80° C., and after the polyvinyl alcohol was completely dissolved, ultrasonic mixing was carried out for 4 h. Subsequently, 1 g of modified molecular sieve was added, and ultrasonicating was carried out until the system is homogeneous. Then, azodiisobutyronitrile was added and thoroughly mixed for 0.5 h to obtain homogeneous liquid.
Embodiment 5: A difference from Embodiment 1 is that:
(3) 3 g of polyvinylidene fluoride 17-88 was placed in 4.5 g of dimethyl sulfoxide and uniformly mixed, and then 0.45 g of polyvinyl alcohol was added. In a nitrogen atmosphere, a system was heated to 80° C., and after the polyvinyl alcohol was completely dissolved, ultrasonic mixing was carried out for 4 h. Subsequently, 1 g of modified molecular sieve was added, and ultrasonicating was carried out until the system is homogeneous. Then, azodiisobutyronitrile was added and thoroughly mixed for 0.5 h to obtain homogeneous liquid.
Embodiment 6: A difference from Embodiment 1 is that:
(3) 3 g of polyvinylidene fluoride 17-88 was placed in 9 g of dimethyl sulfoxide and uniformly mixed, and then 0.45 g of polyvinyl alcohol was added. In a nitrogen atmosphere, a system was heated to 80° C., and after the polyvinyl alcohol was completely dissolved, ultrasonic mixing was carried out for 4 h. Subsequently, 1 g of modified molecular sieve was added, and ultrasonicating was carried out until the system is homogeneous. Then, azodiisobutyronitrile was added and thoroughly mixed for 0.5 h to obtain homogeneous liquid.
Embodiment 7: A difference from Embodiment 1 is that:
(3) 3 g of polyvinylidene fluoride 17-88 was placed in 7 g of dimethyl sulfoxide and uniformly mixed, and then 0.3 g of polyvinyl alcohol was added. In a nitrogen atmosphere, a system was heated to 80° C., and after the polyvinyl alcohol was completely dissolved, ultrasonic mixing was carried out for 4 h. Subsequently, 1 g of modified molecular sieve was added, and ultrasonicating was carried out until the system is homogeneous. Then, azodiisobutyronitrile was added and thoroughly mixed for 0.5 h to obtain homogeneous liquid.
Embodiment 8: A difference from Embodiment 1 is that:
(3) 3 g of polyvinylidene fluoride 17-88 was placed in 7 g of dimethyl sulfoxide and uniformly mixed, and then 0.6 g of polyvinyl alcohol was added. In a nitrogen atmosphere, a system was heated to 80° C., and after the polyvinyl alcohol was completely dissolved, ultrasonic mixing was carried out for 4 h. Subsequently, 1 g of modified molecular sieve was added, and ultrasonicating was carried out until the system is homogeneous. Then, azodiisobutyronitrile was added and thoroughly mixed for 0.5 h to obtain homogeneous liquid.
Comparative Example 1: a difference from Embodiment 1 is that:
(1) 3 g of tetrapropylammonium hydroxide was added into sodium hydroxide solution with a concentration of 0.16 mol/L and uniformly mixed, then 2 g of tetraethyl orthosilicate was added, uniformly mixed, and then stirred for 8 h to obtain a mixture. The mixture was crystallized at 180° C. for 48 h to obtain a product, and then the product was thoroughly washed and dried to obtain a precursor. 1.5 g of the precursor was dispersed in water, and 10 mL of sodium hydroxide solution with a concentration of 0.4 mol/L was added and uniformly mixed, and then 0.25 g of sodium metaaluminate was added, uniformly mixed, and crystallized at 180° C. for 18 h to obtain a product. The product was thoroughly washed, dried, and then calcined at 550° C. for 6 h to obtain a molecular sieve.
Process embodiment: The purification material was placed in a membrane module, a temperature of a feed solution was controlled at 40° C., the feed solution was pumped into and flowed through the membrane module, where a pressure on a permeate side was maintained at −0.4 MPa. After negative pressure permeation was carried out for 2 h, the feed solution was heated to 65° C., and permeation was continued until an ethanol content in the feed solution was below 0.1%. The permeate was then collected.
Process Comparative Example 1: The purification material was placed in a membrane module, a temperature of a feed solution was controlled at 40° C., the feed solution was pumped into and flowed through the membrane module, where a pressure on a permeate side was maintained at 300 Pa. After negative pressure permeation was carried out for 2 h, the feed solution was heated to 85° C., and permeation was continued until an ethanol content in the feed solution was below 0.1%. The permeate was then collected.
Process Comparative Example 2: the purification material was placed in a membrane module, a temperature of a feed solution was controlled at 60° C., the feed solution was pumped into and flowed through the membrane module, where a permeate side was kept under vacuum. Negative pressure permeation was carried out until an ethanol content in the feed solution was below 0.1%. The permeate was then collected.
Sample Testing of Embodiments: A feed solution composed of 90 wt % ethyl formate, 9 wt % ethanol, and 1 wt % water was prepared. Tests were carried out using the purification materials of various embodiments and comparative examples, following procedures in the process examples. The purification material of Embodiment 1 was tested according to Process Comparative Example 1 and Process Comparative Example 2. The mass of substances penetrating through the purification membrane per unit time and per unit membrane area was determined, and a permeate flux was calculated. A separation factor was calculated according to the following formula: separation factor-(the concentration of ethanol and water in the permeate divided by the concentration of ethyl formate in the permeate) divided by (the concentration of ethanol and water in the feed solution divided by the concentration of ethyl formate in the feed solution), with results shown as follows.

| | Permeate flux | | Ethanol content in |
|---|---|---|---|
| | (kg/m² · h) | Separation factor | permeate (%) |
| Embodiment 1 | 1.88 | 4057 | 90.04 |
| Embodiment 2 | 2.03 | 2748 | 89.48 |
| Embodiment 3 | 1.15 | 6204 | 90.12 |
| Embodiment 4 | 2.42 | 2143 | 87.93 |
| Embodiment 5 | 0.98 | 5647 | 90.09 |
| Embodiment 6 | 2.52 | 2016 | 88.41 |
| Embodiment 7 | 1.04 | 5418 | 89.87 |
| Embodiment 8 | 1.76 | 2254 | 88.32 |
| Comparative | 2.12 | 1326 | 86.86 |
| Example 1 | | | |
| Process | 2.64 | 1815 | 86.65 |
| Comparative | | | |
| Example 1 | | | |
| Process | 2.04 | 2587 | 87.64 |
| Comparative | | | |
| Example 2 | | | |

Compared with Embodiment 1, during molecular sieve preparation in Comparative Example 1, more aluminum is involved in displacement, and the permeate flux of the prepared material is increased, but the separation factor decreases significantly, indicating that excessive aluminum for displacement is easy to affect a pore structure of the molecular sieve, leading to an increase in pore size and a decrease in selectivity toward the feed solution to be separated. The decrease of a water content in the permeate also reflects that more ethyl formate enters the permeate. During the modification of the molecular sieve, a coupling agent content in Embodiment 2 is low, while that in Embodiment 3 is high. The permeate flux in Embodiment 2 is higher than that in Embodiment 3, whereas the separation factor in Embodiment 3 is higher. However, compared with Embodiment 1, the increase in permeate flux in Embodiment 2 is insignificant, while the separation factor decreases to a greater extent. In addition, in Embodiment 3, the separation factor has a significant increase, whereas the permeate flux also decreases markedly, indicating that the separation efficiency of both Embodiment 2 and Embodiment 3 is inferior to that of Embodiment 1.

In Embodiment, the content of polyvinylidene fluoride is low, a membrane thickness is reduced, and the permeate flux increases significantly, while the separation factor decreases markedly. In Embodiment 5, a content of dimethyl sulfoxide involved in the preparation is low, the permeate flux decreases significantly, the separation factor increases, but the increase in the separation factor is not very significant compared with that in Embodiment 1. In Embodiment 6, the content of dimethyl sulfoxide is high, the permeate flux increases, but the separation factor decreases significantly, and the water content in the permeate also decreases remarkably. As can be learned from Embodiment 5 and Embodiment 6 in combination that the dimethyl sulfoxide directly affects the porosity and pore size of the purification material. Although more dimethyl sulfoxide increases the porosity and flux, the pore size also increases, leading to the decrease in the separation factor. However, less dimethyl sulfoxide is not conducive to pore formation. In Embodiment 7, less polyvinyl alcohol is involved in the preparation, while in Example 8, more polyvinyl alcohol is involved in the preparation. Although the permeate flux in Embodiment 7 is not as good as that in Embodiment 8, and the separation factor is higher than that in Embodiment 8. However, although the permeate flux in Embodiment 8 is high, the separation factor decreases sharply, indicating that polyvinyl alcohol is beneficial to the stability of pore channels, but too much polyvinyl alcohol may increase the pore size and affect the selectivity of the membrane material.

Compared with Embodiment, 1, the permeate flux in Process Comparative Example 1 increases, but the separation factor decreases significantly. In a relatively high temperature, a space through which molecules pass increases and the molecular motion accelerates, which makes the permeate flux increase, but the passage of ethyl formate is also easier, which relatively weakens permeation advantages of ethanol and water due to accelerated molecular motion, resulting in a decrease in the separation factor. However, there is in stage-wise temperature increase in Process Comparative Example 2. Although the permeate flux in Process Comparative Example 2 is higher than that in Embodiment 1, the separation factor decreases obviously. Compared with Comparative Example 2, Embodiment 1 with stage-wise temperature increase exhibits a significantly greater advantage.

For those skilled in the art, various other corresponding changes and deformations can be made according to the technical solutions and ideas described above, and all these changes and deformations should be within the scope of protection of the claims of the present disclosure.

What is claimed is:

1. A purification material for ethyl formate, wherein the purification material for ethyl formate is prepared by the following procedure: preparing a molecular sieve doped with aluminum on a surface, and carrying out surface grafting modification of the molecular sieve with γ-methacryloxypropyltrimethoxysilane to obtain a modified molecular sieve;
placing polyvinylidene fluoride in dimethyl sulfoxide and mixing uniformly, and then adding polyvinyl alcohol; in a nitrogen atmosphere, heating a system until polyvinyl alcohol is completely dissolved, and ultrasonically mixing for 3 to 5 h; then adding the modified molecular sieve, ultrasonicating until the system is homogeneous, adding azodiisobutyronitrile, and mixing thoroughly for 0.5 h to 1 h to obtain homogeneous solution; and
casting the homogeneous solution onto a matrix to form a film, allowing infiltration by standing for 1 h to 2 h, then freezing to obtain a frozen membrane material; fully replacing dimethyl sulfoxide in the frozen membrane material with water, and drying a wet membrane material after completion of displacement with hot air at 70° C. to 85° C.; and after drying, thoroughly washing the membrane material with hot water, and drying the washed membrane material to obtain a purification material.

2. The purification material for ethyl formate according to 1, wherein the preparation of the molecular sieve comprises the following steps:
a. placing tetrapropylammonium hydroxide in a sodium hydroxide solution and mixing uniformly, then adding tetraethyl orthosilicate and mixing uniformly, and stirring for 6 h to 10 h; after carrying out crystallization for 48 h to 72 h, thoroughly washing a product, and drying the washed product to obtain a precursor;
b. dispersing the precursor in water, adding the sodium hydroxide solution and mixing thoroughly, adding sodium metaaluminate and mixing uniformly, and then carrying out crystallization for 10 h to 24 h; thoroughly washing a product, drying, and then calcining the product at 500° C. to 600° C. for 5 h to 8 h to obtain the molecular sieve.

3. The purification material for ethyl formate according to claim 1, wherein in step a, a concentration of the sodium hydroxide solution ranges from 0.15 mol/L to 0.17 mol/L; and a mass ratio of the tetrapropylammonium hydroxide to the tetraethyl orthosilicate is 1.2 to 1.5:1.

4. The purification material for ethyl formate according to claim 3, wherein in step b, a mass ratio of the precursor to sodium hydroxide is 1:0.08 to 0.15; and a mass ratio of the precursor to the sodium metaaluminate is 1:0.08 to 0.12.

5. The purification material for ethyl formate according to claim 3, wherein crystallization is carried out at 160° C. to 190° C.

6. The purification material for ethyl formate according to claim 1, wherein a mass ratio of the molecular sieve to the γ-methacryloxypropyltrimethoxysilane is 1:0.15 to 0.25; a mass ratio of the modified molecular sieve to the polyvinylidene fluoride is 1:2 to 4; a mass ratio of the polyvinylidene fluoride to the dimethyl sulfoxide is 1:1.5 to 3; and a mass ratio of the polyvinylidene fluoride to the polyvinyl alcohol is 1:0.1 to 0.2.

7. The purification material for ethyl formate according to claim 1, wherein the freezing is carried out at a temperature below −15° C. for 8 h to 12 h.

8. A purification process for ethyl formate using the purification material according to claim 1, comprising the following steps: loading the purification material into a membrane module, and controlling a feed solution at 35° C. to 45° C.; and after carrying out negative pressure permeation for 1 h to 3 h, heating the feed solution to below 60° C., and collecting permeate during the process.

9. The purification process for ethyl formate according to claim 8, wherein a feed flow rate ranges from 10 mL/min to 50 mL/min, the membrane is taken out and treated at 50° C. to 80° C. under −0.08 MPa to −0.04 MPa for 1 h to 3 h to complete regeneration.
